(19) United States
(12) Patent Application Publication
(10) Pub. No.: US 2018/0198321 A1
(43) Pub. Date: Jul. 12, 2018

(54) Multiphysics Energy Harvester for Implants

(71) Applicant: QUALCOMM Incorporated, San Diego, CA (US)

(72) Inventor: Arvind Govindaraj, San Diego, CA (US)

(21) Appl. No.: 15/400,746

(22) Filed: Jan. 6, 2017

(51) Int. Cl. H02J 50/30; H02J 50/15; H02N 2/18; A61N 1/378; A61B 5/00

(52) U.S. Cl. CPC H02J 50/30; H02J 50/15; A61B 2560/0219; A61N 1/3787; A61B 5/686; H02N 2/186

(57) ABSTRACT

The present disclosure describes aspects of a multiphysics energy harvester for implants. In some aspects, an apparatus includes a multiphysics energy (MPE) harvester to harvest energy from at least a first and second type of wireless power transfer signal. The MPE harvester includes a first harvesting component configured to react to the first type of wireless power transfer signal, effective to harvest energy from the first type of wireless power transfer signal. The MPE harvester also includes a second harvesting component that is integral with the first harvesting component. The second harvesting component is configured to react to the second type of wireless power transfer signal simultaneously as the first harvesting component reacts to the first type of wireless power transfer signal. The reactions of the second harvesting component are effective to harvest energy from the second type of wireless power transfer signal.

### FIELD OF THE DISCLOSURE

This disclosure relates generally to wirelessly transferring power to implants within a body. More particularly, the disclosure relates to harvesting energy from multiple different wireless power transfer signals.

### BACKGROUND

This description of related art is provided for the purpose of generally presenting a context for the disclosure that follows. Unless indicated otherwise herein, concepts described in this section are not prior art to this disclosure and are not admitted to be prior art by inclusion herein.

Biomedical implants are becoming more common for treatment of disease and medical conditions in humans as well as in animals. These implants can be inserted into a host's body for a variety of purposes, such as to release metered doses of medication, stimulate bodily tissue (e.g., nerves), monitor specific biochemical conditions, and so on. Oftentimes, such implants require electrical energy in order to operate—they need a power source, which typically takes the form of a chemical battery. Although implants are expected to be operative for several years (or a host's lifetime) without replacement, the chemical batteries used to power them may not be capable of operating that long. Thus, to keep these implants operating as designed, their batteries may need to be changed. Changing chemical batteries that are implanted can be difficult, however, and doing so can pose a significant risk to the host. Accordingly, conventional techniques for powering implants can put a host's life at risk.

### SUMMARY

In some aspects of a multiphysics energy harvester for implants, an apparatus is capable of harvesting energy simultaneously from multiple different types of wireless power transfer signals to power an implant. The apparatus includes a multiphysics energy (MPE) harvester to harvest energy from at least a first and second type of wireless power transfer signal. In particular, the MPE harvester includes a first harvesting component that is configured to react to the first type of wireless power transfer signal, effective to harvest the energy from the first type of wireless power transfer signal. The MPE harvester also includes a second harvesting component that is integral with the first harvesting component. The second harvesting component is configured to react to the second type of wireless power transfer signal simultaneously as the first harvesting component reacts to the first type of wireless power transfer signal. The reactions of the second harvesting component to the second type of wireless power transfer signal are effective to harvest the energy from the second type of wireless power transfer signal.

Some aspects of a multiphysics energy harvester for implants also involve a method in which energy is harvested based on reactions of a first harvesting component of a multiphysics energy (MPE) harvester within a body to a first type of wireless power transfer signal. The method also comprises simultaneously harvesting additional energy based on reactions of a second harvesting component of the MPE harvester to a second type of wireless power transfer signal, which is different from the first type of wireless power transfer signal. In accordance with the described aspects, the second harvesting component is integral with the first harvesting component. Further, the method comprises converting the harvested energy and the additional harvested energy into electrical power that is usable to power an electronic device, such as an implant.

In other aspects, a method for configuring a system to harvest energy from multiple different types of wireless power transfer signals simultaneously to power an implant comprises disposing a first harvesting component in a multiphysics energy (MPE) harvester. In accordance with the described aspects, the first harvesting component is configured to harvest energy based on reactions to a first type of wireless power transfer signal. The method also comprises disposing a second harvesting component in the MPE harvester integral with the first harvesting component. The second harvesting component is configured to simultaneously, while the first harvesting component harvests energy, harvest additional energy based on reactions to a second type of wireless power transfer signal. In one or more aspects, the second type of wireless power transfer signal is different from the first type of wireless power transfer signal. Further, the method comprises coupling power-conversion circuitry with the MPE harvester to convert and combine the harvested energy and the additional harvested energy into electrical power that is usable by the implant.

In some aspects, an apparatus for powering an implant using energy harvested simultaneously from multiple different types of wireless power transfer signals includes a first harvesting means for harvesting energy from a first type of wireless power transfer signal. The apparatus also includes a second harvesting means that is integral with the first harvesting means for simultaneously harvesting additional energy from a second type of wireless power transfer signal. Further, the apparatus includes a power generation means for generating power that is usable by the implant and is generated from the energy and the additional energy harvested by the first and second harvesting means, respectively.

### BRIEF DESCRIPTION OF DRAWINGS

The details of various aspects are set forth in the accompanying figures and the detailed description that follows. In the figures, the left-most digit of a reference number identifies the figure in which the reference number first appears. The use of the same reference numbers in different instances in the description or the figures indicates like elements:

FIG. 1 illustrates an example environment that includes a multiphysics energy (MPE) harvester to power an implant.

FIG. 2 illustrates an example configuration of the MPE harvester shown in FIG. 1.

FIG. 3 illustrates an example block diagram for an implant with power-conversion circuitry coupled to harvesting components of the MPE harvester shown in FIG. 1.

FIG. 4 illustrates an example implementation of an MPE harvester configured as a cantilever and proof mass.

FIG. 5 illustrates an example method for harvesting energy from multiple different wireless power transfer signals using an MPE harvester.

FIG. 6 illustrates an example method for configuring a system to harvest energy, for powering an implant, from multiple different wireless power transfer signals.

FIG. 7 illustrates a system-on-chip (SoC) having components through which aspects of an MPE harvester can be implemented.

### DETAILED DESCRIPTION

Devices implanted in humans and animals are becoming more common, such as biomedical implants capable of treating disease and medical conditions. As used herein, a “host” refers to a respective body (e.g., human or animal) in which an implant is surgically inserted. Biomedical implants can be inserted into a host's body for a variety of purposes as described above and below. Many implants (biomedical or otherwise) often require electrical energy in order to operate. In other words, these implants need a power source. Often the power source used to power an implant is a chemical battery. Broadly speaking, implants are capable of operating for several years (or a host's entire lifetime) without replacement. The chemical batteries used to power these implants, however, often are not capable of providing power that long. Thus, to keep an implant operating as designed, its battery may need to be surgically changed. The surgical procedures for changing implanted chemical batteries can be invasive and difficult to perform, however. Furthermore, doing so can pose a significant risk to the host. Accordingly, conventional techniques for powering implants can put a host's life at risk.

Further, some conventional wireless charging techniques may not be suitable for implanted electronic devices having small form factors, e.g., 1 cm or less. At this size, for instance, conventional wireless charging techniques involving electromagnetic waves may be unsuitable due to specific absorption rate (SAR) limits—SAR limits are regulatory-defined limits on the rates at which devices are allowed to expose a human body to radio frequency electromagnetic fields—as well as due to attenuation and directivity of the electromagnetic waves. Conventional wireless charging techniques involving acoustic energy may be unsuitable due to cavitation caused by the acoustics and due to directivity. Some such acoustic-wireless charging techniques may also exhibit inefficiencies as a result of anchoring energy harvesting components to structures within a body. Delivering an amount of energy needed to power an implant may also be difficult using conventional infrared-wireless charging techniques due to attenuation of the infrared by the body.

This disclosure describes aspects of wirelessly charging an implant using power harvested by a three-dimensional (3D) multiphysics transducer, which is referred to herein as a multiphysics energy harvester (MPE harvester). Unlike conventional techniques, the MPE harvester is used to harvest power simultaneously from multiple different wireless power transfer signals. Examples of wireless power transfer signals include electromagnetic waves, acoustic energy (e.g., ultrasound), and infrared, although other wireless power transfer signals may be leveraged herein without departing from the spirit or scope of the described techniques.

To harvest power from various types of wireless power transfer signals, the MPE harvester combines multiple components, each of which may be capable of harvesting power from one type of wireless power transfer signal. By way of example, the MPE harvester may combine a piezoelectric structure and a photodiode, where the piezoelectric structure is capable of harvesting energy from acoustic waves and the photodiode is capable of harvesting energy from infrared signals. Further, the MPE harvester may include or be coupled to circuitry capable of rectifying an alternating current (AC) signal induced in one of the components (e.g., a piezoelectric structure) to produce direct current (DC) power and combining this DC power with a DC signal from another one of the components (e.g., a photodiode). The apparatuses and methods described herein may generate power to charge an implant's battery utilizing the energy harvested from these signals.

By harvesting energy simultaneously from multiple different wireless power transfer signals, many of the drawbacks of conventional techniques can be avoided. For example, the MPE harvester may have a small form factor, e.g., 1 cm or less, yet still supply an implant with enough power to operate using wireless charging techniques that expose hosts to electromagnetic fields at rates below SAR limits. The implant may be supplied with enough power because the described techniques do not rely solely on the electromagnetic fields for wireless charging. Instead, the described techniques are capable of combining energy harvested from electromagnetic fields with energy harvested simultaneously from a different type of wireless power transfer signal. It is by harvesting energy simultaneously from multiple different wireless power transfer signals that the MPE harvester is also capable of supplying an implant with enough power to operate despite power loss due to attenuation and directivity of a wireless signal, as well as inefficiencies of individual harvesting components.

These and other aspects of an MPE harvester for implants are described below in the context of an example environment, example MPE harvesters, and techniques. Any reference made with respect to the example environment or MPE harvester, or elements thereof, is by way of example only and is not intended to limit any of the aspects described herein.

### Example Environment

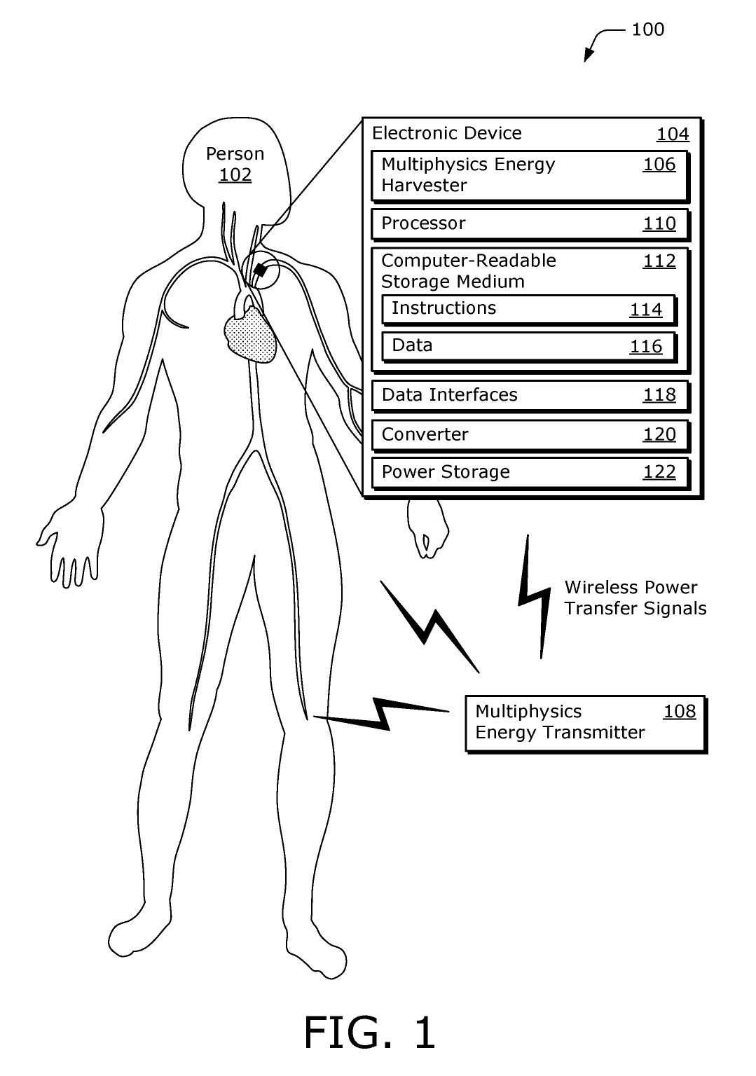

FIG. 1 illustrates an example environment 100, which includes a person 102 in which an implanted electronic device 104 has been surgically inserted. In this example, the electronic device 104 includes a multiphysics energy harvester 106 (MPE harvester 106) though in some aspects the MPE harvester 106 may be implanted in the person 102 and not incorporated as part of the electronic device 104—the MPE harvester 106 may be remote from the electronic device 104. In such aspects, the MPE harvester 106 may instead simply be coupled (e.g., via a wired or wireless coupling) to the electronic device 104. The example environment also includes a multiphysics energy transmitter 108 (MPE transmitter 108), which is configured to transmit multiple different types of wireless power transfer signals to supply power wirelessly to the electronic device 104.

The electronic device 104 may be implemented as any suitable computing or electronic device that is implanted in the person 102 and capable of being powered with power harvested by the MPE harvester 106 from the multiple different types of wireless power transfer signals transmitted by the MPE transmitter 108. Examples of the electronic device 104 include implants to release metered doses of medication, implants to stimulate bodily tissue (e.g., nerves), implants for managing reproduction, implants to monitor specific biochemical conditions, and so on. Electronic devices other than medical-based implants may also be contemplated within the techniques described herein, such as personal communication devices, identification devices, location tracking devices, and so forth. Accordingly, the electronic device 104 may correspond to a variety of different implanted computing or electronic devices without departing from the spirit or scope of the techniques described herein.

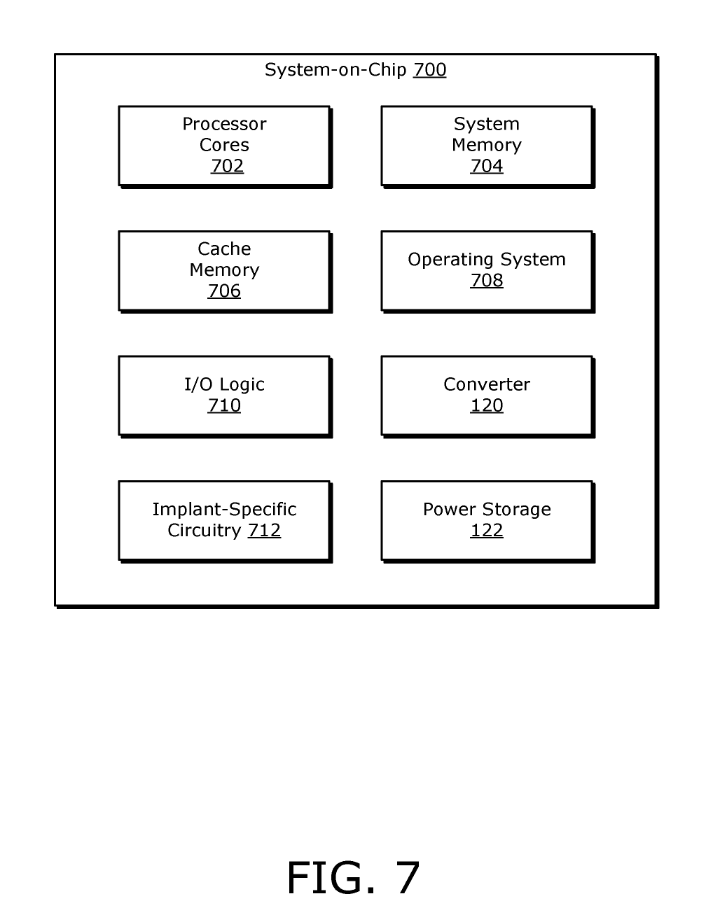

The electronic device 104 includes a processor 110. In the example, the electronic device 104 also includes computer-readable storage medium 112 (CRM 112). The processor 110 may include any type of processor, such as an application processor or multi-core processor, configured to execute processor-executable code stored by the CRM 112. The CRM 112 may include any suitable type of data storage media, such as volatile memory (e.g., random access memory (RAM)), non-volatile memory (e.g., Flash memory), optical media, magnetic media (e.g., disk or tape), and the like. In the context of this disclosure, the CRM 112 is implemented to store instructions 114, data 116, and other information of the electronic device 104, and thus does not include transitory propagating signals or carrier waves. Further, although the electronic device 104 is illustrated with the CRM 112, in some aspects the electronic device 104 may instead or additionally be implemented using a system-on-chip (SoC) as further described in relation to FIG. 7.

In the example, the electronic device 104 also includes data interfaces 118. The data interfaces 118 provide connectivity to respective networks and other electronic devices connected therewith. The data interfaces 118 may comprise wired data interfaces (that are usable to connect with the electronic device 104 before it is implanted into a body, during a surgical procedure in which the electronic device 104 is exposed, when the electronic device 104 has been removed from the body, and so on), wireless data interfaces, or any suitable combination thereof. Alternately or additionally, the wireless interfaces may include a modem or radio configured to communicate over a wireless network, such as a wireless LAN, peer-to-peer (P2P), cellular network, and/or wireless personal-area-network (WPAN).

The electronic device 104 also includes converter 120 and power storage 122. The converter 120 represents functionality to convert power generated using the energy harvested with the MPE harvester 106 into a form that is usable by the electronic device 104. This power enables the electronic device 104 to perform its corresponding functionality, e.g., release metered doses of medication and so on. By way of example, the converter 120 represents functionality to boost a voltage of the power generated.

In some scenarios, the MPE harvester 106 may generate more power than is usable by the electronic device 104 at the time. To store this excess power, the power storage 122 may be used. The power storage 122 represents functionality to store power generated using energy harvested with the MPE harvester 106 for later use. For instance, the power storage 122 may be configured as a type of battery. In some aspects, the converter 120 may feed the power storage 122, and the electronic device 104 may draw power for operation from the power storage 122. In other aspects, the electronic device 104 may draw power for operation directly from the converter 120 and rely on the power storage 122 solely when the power supplied directly from the converter 120 is not enough to function properly. In both cases, the electronic device 104 is configured to use power stored in the power storage 122 for operation.

The MPE transmitter 108 represents functionality to transmit multiple different types of wireless power transfer signals. The MPE transmitter 108 may be configured as an apparatus and/or multiple apparatuses that, from outside the person 102, transmit the multiple different types of wireless power transfer signals, which pass through bodily tissue of the person 102 and eventually reach the MPE harvester 106. For example, the MPE transmitter 108 may be placed on the person 102's skin or on a bedside table and transmit at least two different types of wireless power transfer signals for receipt by the MPE harvester 106. The MPE transmitter 108 may be capable of transmitting any of a variety of different wireless power transfer signals, including electromagnetic waves, acoustic energy (e.g., ultrasound), and infrared. Indeed, the MPE transmitter 108 may be configured to transmit other wireless power transfer signals as well as a variety of different combinations of wireless power transfer signals without departing from the spirit or scope of the techniques described herein.

In general, the MPE harvester 106 represents functionality to harvest energy simultaneously from multiple different wireless power transfer signals. The MPE harvester 106 may be configured as an apparatus and/or multiple interoperable components that are surgically implanted into the person 102 and capable of leveraging multiple different types of wireless power transfer signals to provide power to the electronic device 104. In particular, the multiple different types of wireless power signals are leveraged to provide more power than approaches that leverage a single type of wireless power transfer signal. To do so, multiple components, each of which is capable of harvesting energy from a given type of wireless power signal, are combined to form the MPE harvester 106.

By way of example, the MPE harvester 106 can include a component that is capable of harvesting energy when exposed to acoustic signals and is integral with another component capable of harvesting energy when exposed to infrared signals. Broadly speaking, acoustic waves can be converted to electrical energy using various different piezo structures formed from piezoelectric materials, using di-electric elastomers, and so on. Infrared signals can be converted to electrical energy using various light-conversion components, such as photodiodes. Although harvesting energy from acoustic and infrared signals is described, the MPE harvester 106 can include components capable of harvesting energy from other types of wireless power transfer signals, such as electromagnetic waves, different wavelengths of light, and so forth.

Regardless of the particular types of wireless power transfer signals leveraged, the components of the MPE harvester 106 can be configured as microelectromechanical systems (MEMS) to use in conjunction with an electronic device 104 having a small form factor. Typically, MEMS components range in size from 20 micrometers to 1 micrometer. Continuing with the example in which acoustic and infrared signals are leveraged, the component capable of harvesting the acoustic signals (e.g., a piezo structure) can be 20 micrometers to 1 millimeter in size. Similarly, the component capable of harvesting the infrared signals (e.g., a photodiode) can also be 20 micrometers to 1 millimeter in size. In some aspects, the MPE harvester 106 can be configured with arrays of MEMS-scale components for harvesting energy. As described below, the individual components can be integrated into a single component forming the MPE harvester 106.

In accordance with one or more aspects, the MPE transmitter 108 and at least one of the MPE harvester 106 or the electronic device 104 (e.g., the data interfaces 118) may include functionality to communicate to control the power transferred using the MPE transmitter 108. The MPE harvester 106, for instance, may communicate an indication to the MPE transmitter 108 that indicates to increase an amount of power, e.g., by increasing a strength of one or more of the wireless power transfer signals. Similarly, the MPE harvester 106 may communicate an indication to the MPE transmitter 108 to decrease the amount of power or cease power transfer, e.g., by decreasing a strength or ceasing transmission of one or more of the wireless power transfer signals. This communication may be carried out in a variety of different ways, including using either or both of in-band or out-of-band communication techniques. How an MPE harvester 106 may be specifically implemented to generate power using energy harvested from multiple different wireless power transfer signals is described in greater detail below.

### Example Multiphysics Energy Harvesters

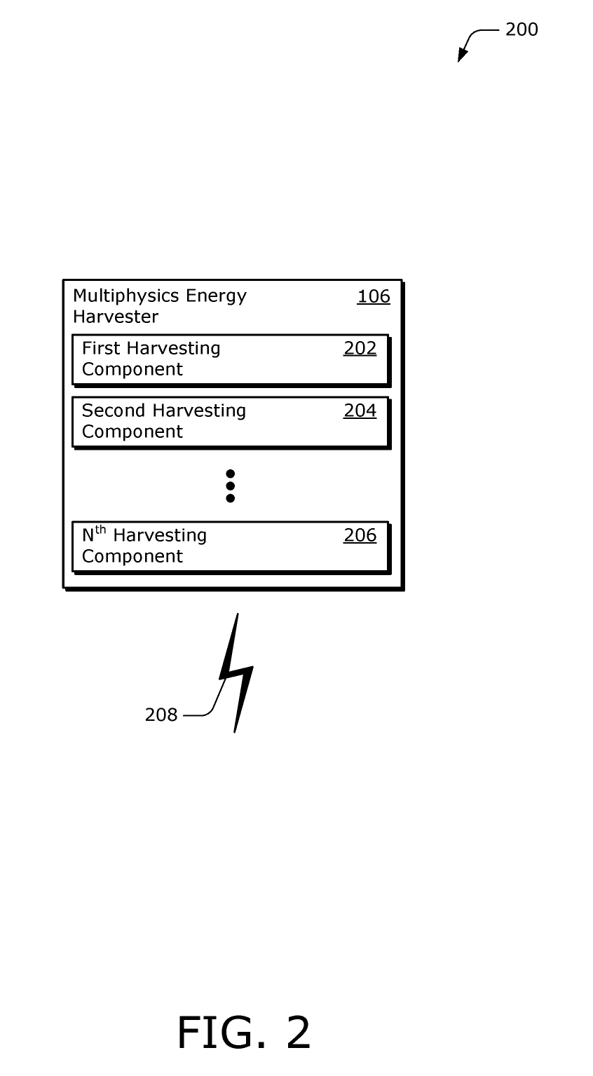

FIG. 2 illustrates an example configuration of the multiphysics energy harvester 106 (MPE harvester 106) from FIG. 1 in accordance with one or more aspects at 200. The MPE harvester 106 is depicted having first harvesting component 202 and second harvesting component 204. This represents that in at least some implementations, the MPE harvester 106 may be configured with components to harvest energy from just two different types of wireless power transfer signals. In the illustrated example, however, the MPE harvester 106 also includes N^{th} harvesting component 206. The N^{th} harvesting component 206 represents that the MPE harvester 106 may be configured to harvest energy from more than two different types of wireless power transfer signals in some implementations. These components of the MPE harvester may be integrated with one another as described above and below, e.g., the second harvesting component 204 may be attached to the first harvesting component 202 in a variety of different ways.

In general, the first harvesting component 202 is configured to react to a first type of wireless power transfer signal. The MPE harvester 106 converts these reactions to the first type of wireless power transfer signal into electrical power that can be used to power the electronic device 104. By way of example, the first harvesting component 202 may be configured to react to one of electromagnetic waves, acoustic energy (e.g., ultrasound), or infrared. With reference to the example environment 100, this first type of wireless power transfer signal may be transmitted by the multiphysics energy transmitter 108 (MPE transmitter 108).

In accordance with the described aspects, the second harvesting component 204 is configured to react to a second type of wireless power transfer signal that is different from the first type of wireless power transfer signal. For instance, the second harvesting component 204 reacts to a different one of electromagnetic waves, acoustic energy (e.g., ultrasound), or infrared than the first harvesting component 202. In any case, MPE harvester 106 is configured to convert these reactions of the second harvesting component 204 to the second type of wireless power transfer signal into electrical power that can be used to power the electronic device 104. Like the first type of wireless power transfer signal, the second type of wireless power transfer signal may also be transmitted by the MPE transmitter 108.

The N^{th} harvesting component 206 may be configured to react to a same type of signal as the first or second harvesting components 202, 204, respectively, or may be configured to react to a different, third type of wireless power transfer signal. The MPE harvester 106 can convert the N^{th} harvesting component 206's reactions to the corresponding signal into electrical power that can be used to power the electronic device 104. Regardless of the particular type of wireless power transfer signal leveraged by the N^{th} harvesting component 206, the MPE transmitter 108 can be configured to transmit more than two types of wireless power transfer signals. The MPE transmitter 108 may be configured to transmit N-number of wireless power transfer signals, for example. In some aspects, the number of different types of wireless power transfer signals the MPE transmitter 108 is configured to transmit may be the same as the number of components with which the MPE harvester 106 is configured to harvest different types of wireless power transfer signals. In other aspects, the MPE transmitter 108 may be configured to transmit a number of wireless power transfer signals that is greater or less than the number of components with which the MPE harvester 106 is configured to harvest different types of wireless power transfer signals. For instance, the MPE transmitter 108 may be configured to transmit three different types of wireless power transfer signals while the MPE harvester 106 includes components configured to harvest energy from two of those signals.

The illustrated example also includes power coupling 208, which represents functionality to transfer power generated by the MPE harvester 106 to the electronic device 104. As discussed above, the MPE harvester 106 may be incorporated as part of the electronic device 104. In such scenarios, the power coupling 208 may also be implemented as part of the electronic device 104, e.g., as a wired coupling in the electronic device 104 between the MPE harvester 106 and the converter 120 or the power storage 122. In some scenarios where the MPE harvester 106 is part of the electronic device 104 the power coupling 208 may be a wireless coupling, e.g., a wireless coupling to a circuit coupled to the converter 120 or the power storage 122. Alternately, the MPE harvester 106 may not be incorporated within the electronic device 104. In these scenarios, the power coupling 208 may also be implemented as a wired or wireless coupling, e.g., between the MPE harvester 106 and the electronic device 104.

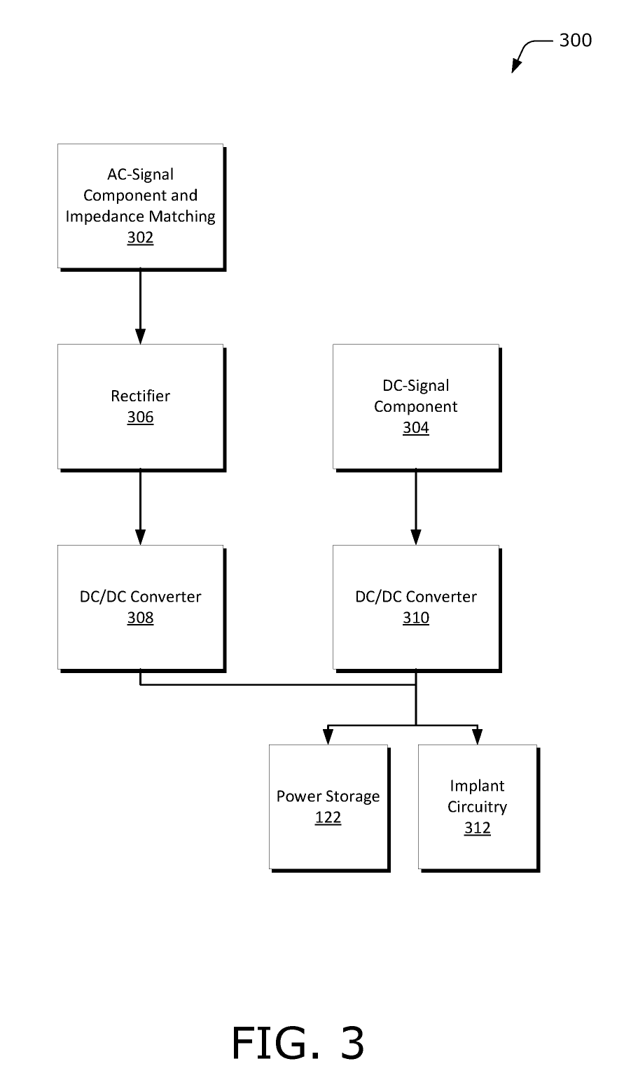

FIG. 3 illustrates an example block diagram for an implant with power-conversion circuitry coupled to harvesting components of the multiphysics energy harvester 106 (MPE harvester 106) from FIG. 1 in accordance with one or more aspects at 300.

The example block diagram 300 includes AC-signal component and impedance matching 302 and DC-signal component 304, which represent harvesting components of the MPE harvester 106. By way of example, the AC-signal component and impedance matching 302 may correspond to a piezoelectric component, such as a piezoelectric cantilever. In general, the AC-signal component and impedance matching 302 represents functionality to produce an AC signal at the MPE harvester 106 when exposed to certain types of transmitted energy, such as acoustic energy. In contrast, the DC-signal component 304 represents functionality to produce a DC signal at the MPE harvester 106 when exposed to other types of transmitted energy, such as infrared. In accordance with one or more aspects, the DC-signal component 304 corresponds to a photodiode, though other components capable of producing a DC signal may be used without departing from the spirit or scope of the techniques described herein.

In addition, the example block diagram 300 includes circuitry capable of converting and combining the energy harvested using the AC-signal component and impedance matching 302 and the DC-signal component 304 so that it is usable by an implant. In particular, the example block diagram 300 depicts rectifier 306, and DC/DC converters 308, 310. Combinations of these components may be referred to herein as “power-conversion circuitry.” The rectifier 306 represents functionality to rectify the AC signal produced by the AC-signal component and impedance matching 302 to DC. In accordance with one or more aspects, the rectifier 306 may be implemented using field-effect transistors (FETs).

The DC/DC converters 308, 310 represent functionality to convert the DC signals produced by the rectifier 306 and the DC-signal component 304, respectively. In general, the DC/DC converters 308, 310 represent functionality to translate a voltage range (e.g., at the output of the rectifier 306 or output of the DC-signal component 304, respectively) to match a voltage range of the power storage 122 or implant circuitry 312. The implant circuitry 312 represents structures of an implant for carrying out its corresponding functionality.

The DC/DC converters 308, 310 may, for example, be capable of up-converting the DC-signals to provide power that is usable by implant circuitry 312. In addition or alternately, the DC/DC converters 308, 310 may up-convert the DC-signals to charge the power storage 122. Although the block diagram is illustrated having the DC/DC converters 308, 310, in accordance with one or more aspects, these converters may be optional. In other words, some implants that leverage MPE harvesters (or the MPE harvesters themselves) may not include the DC/DC converters 308, 310. Further still, some aspects may involve configurations including one, but not both, of the DC/DC converters 308, 310.

As discussed above, the power storage 122 also may not be included in some implementations, e.g., where power is provided directly to the implant circuitry 312 without first being stored. Nonetheless, the DC-signal component 304 can be wired in a series or parallel structure to match its optimal voltage range to a voltage range of battery variations. Similarly, the AC-signal component and impedance matching 302 may be wired to match a voltage range of a battery.

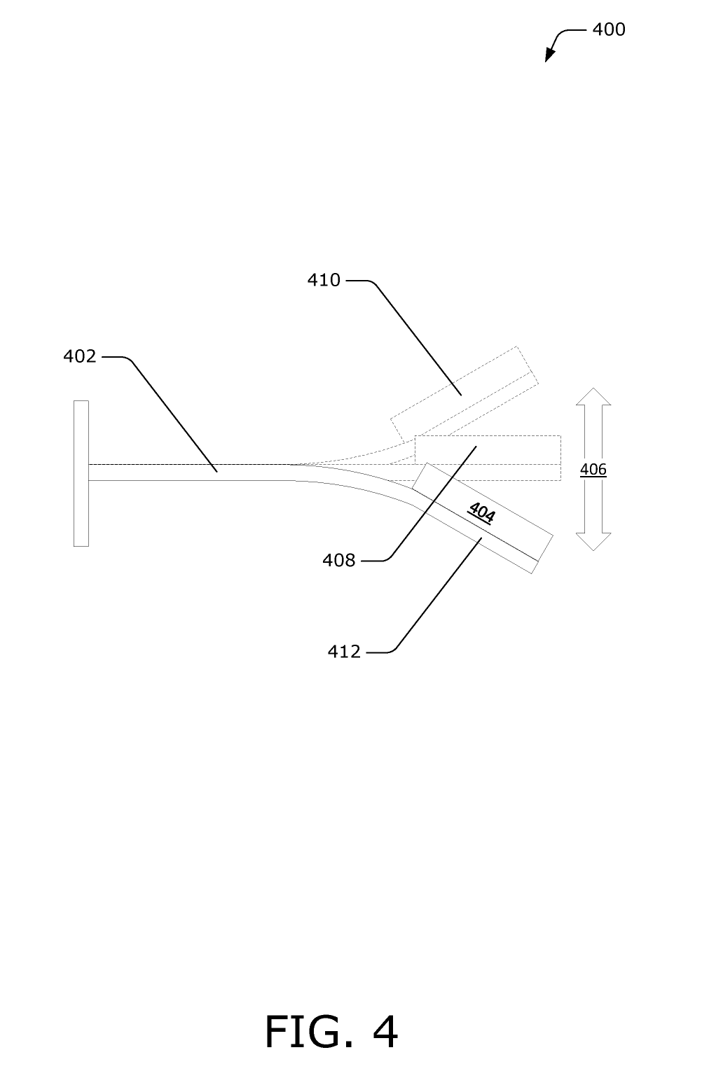

FIG. 4 illustrates an example implementation of an MPE harvester configured as a cantilever and proof mass in accordance with one or more aspects at 400. In particular, the illustrated example depicts piezo cantilever 402 and photodiode 404, which is stacked on the piezo cantilever 402 to serve as a proof mass.

When exposed to the influence of external accelerations, such as ultrasound signals, the photodiode 404 as proof mass may deflect 406 the piezo cantilever 402 from a neutral position 408. The external accelerations may cause the piezo cantilever 402 to oscillate between extreme positions 410, 412, for example. Oscillation of the piezo cantilever 402 is effective to induce a voltage, e.g., an alternating voltage (AC) signal with a period that corresponds to the oscillatory period of the piezo cantilever 402's oscillation. In this way, the piezo cantilever 402 is used to harvest energy from wireless power transfer signals that cause the photodiode 404 to deflect the piezo cantilever 402 from the neutral position 408. By way of example, those wireless power transfer signals include acoustic signals, such as ultrasound.

Although the photodiode 404 is configured to serve as a proof mass for the piezo cantilever 402 of the illustrated example, the photodiode 404 is also configured to harvest energy independently of the piezo cantilever 402. In particular, the photodiode 404 is configured to harvest energy from a different type of wireless power transfer signal than the piezo cantilever 402. Not only is the photodiode 404 configured to harvest energy from a different type of wireless power transfer signal, but it is capable of doing so simultaneously while the piezo cantilever 402 harvests energy from its respective type of wireless power transfer signal. The photodiode 404 may, for instance, harvest energy from electromagnetic radiation signals such as infrared. When exposed to such signals, the photodiode 404 absorbs photons and generates a current.

In one or more aspects, the piezo cantilever 402 and the photodiode 404 can be configured as microelectromechanical systems (MEMS) components. In this way, the piezo cantilever 402 and the photodiode 404 can be used in conjunction with an implant having a small form factor. In MEMS implementations, each of the piezo cantilever 402 and the photodiode 404 may thus range in size from 20 micrometers to 1 millimeter. Additionally or alternately, arrays of MEMS-scaled components may be incorporated into the MPE harvester 106 in accordance with one or more aspects.

Further, the MPE harvester 106 may be formed using different components than the piezo cantilever 402 and the photodiode 404 of the illustrated example. Indeed, the MPE harvester 106 may be configured to harvest energy from acoustic waves using a variety of different piezo structures, di-electric elastomers, and so on. Different piezo cantilever arrays, a piezo disk, or a piezo diaphragm may be used in some aspects, for example. In such aspects, a component capable of harvesting energy from electromagnetic radiation signals, such as infrared, may be integrated with the piezo structure. By way of example, a photodiode may be attached to a piezo disk.

Regardless of the particular components chosen, the chosen components can be integrated using a variety of different techniques, including using a semiconductor process, bonding the components, and so on. Referring back to the example depicted in FIG. 4, the photodiode 404 can be integrated on top of the piezo cantilever 402 to form the MPE harvester 106 using a semiconductor process, by bonding the photodiode 404 with the piezo cantilever 402, and so forth. Advantages of combining the photodiode 404 and the piezo cantilever 402 include increasing power reception capabilities of the MPE harvester 106 relative to either the photodiode 404 and the piezo cantilever 402 alone. Another advantage is that the described techniques may less susceptible to variations in power generation due to a location and orientation of the MPE harvester 106 within the person 102.

It should be appreciated that the MPE harvester 106 can be configured to harvest energy from a variety of different combinations of wireless power transfer signals, which include combinations involving acoustic and/or infrared signals, like the example illustrated in FIG. 4, as well as other combinations of wireless power transfer signals. Further, the MPE harvester 106 may be configured with different components than illustrated in FIG. 4 when harvesting energy from acoustic and infrared signals without departing from the spirit or scope of the techniques described herein.

### Techniques of Multiphysics Energy Harvesters for Implants

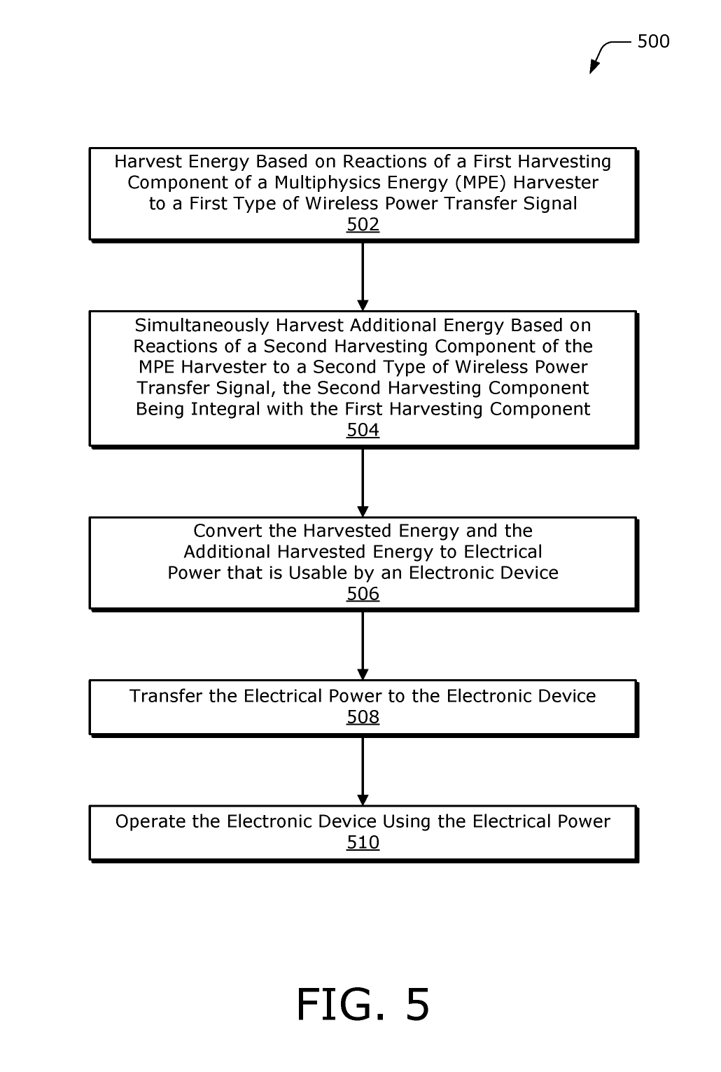

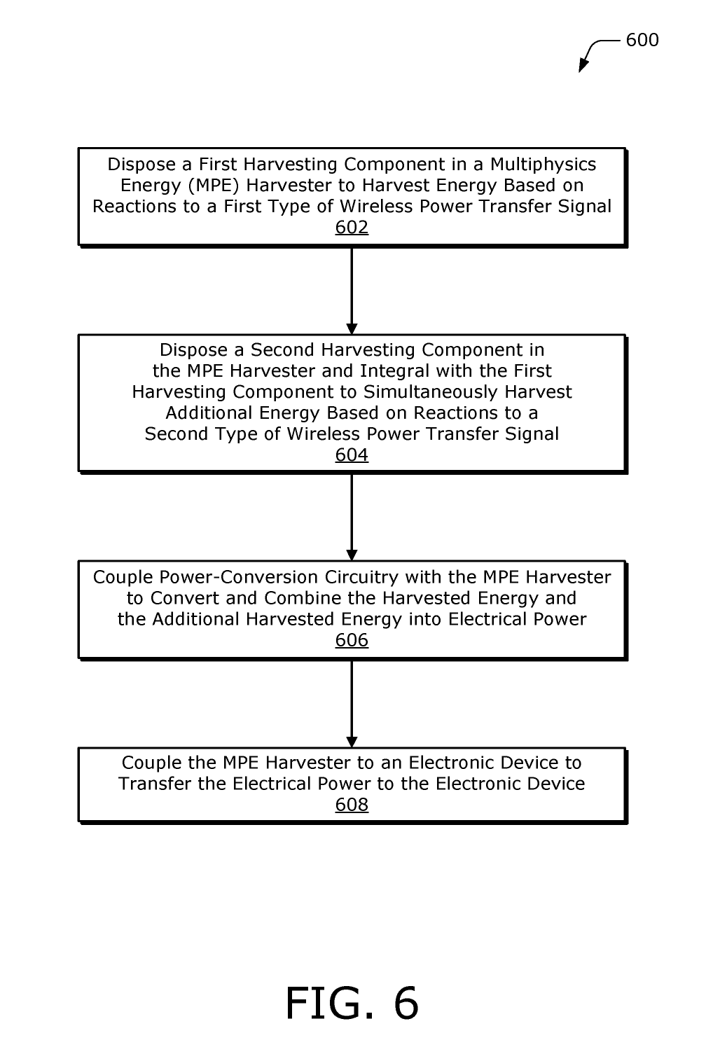

The following techniques of multiphysics energy harvesting for implants may be implemented using any of the previously described multiphysics energy harvesters of the example environment. The techniques may also involve powering an implant configured like the electronic device 104 of the example environment or the system-on-chip described with reference to FIG. 7. Reference to entities, such as the MPE harvester 106 or the electronic device 104, is made by example only and is not intended to limit the ways in which the techniques can be implemented. The techniques are described with reference to example methods illustrated in FIGS. 5 and 6. The example methods are depicted as respective sets of operations or acts that may be performed using the entities described herein and/or any suitable components which provide means for implementing one or more of the operations. The depicted sets of operations illustrate a few of the many ways in which the techniques may be implemented. As such, operations of a method may be repeated, combined, separated, omitted, performed in alternate orders, performed concurrently, or used in conjunction with another method or operations thereof.

FIG. 5 illustrates an example method 500 of harvesting energy from multiple different wireless power transfer signals using a multiphysics (MPE) harvester, including operations performed by the MPE harvester 106. In the following discussion, the MPE harvester 106 or other entities of the example environment 100 may provide means for implementing one or more of the operations described.

At 502, the method includes harvesting energy based on reactions of a first harvesting component of a multiphysics energy (MPE) harvester to a first type of wireless power transfer signal. By way of example, consider FIG. 2, which illustrates the MPE harvester 106 of FIG. 1 in more detail in accordance with one or more aspects generally at 200. In this example, the first harvesting component 202 of the MPE harvester 106 reacts to a first type of wireless power transfer signal. The reactions of the first harvesting component 202 are effective to generate current at the MPE harvester 106—thereby harvesting energy from the first type of wireless power transfer signal to generate power. In accordance with one or more aspects, the MPE transmitter 108 transmits the first type of wireless power transfer signal, which passes through bodily tissue of the person 102 to reach the first harvesting component 202.

At 504, the method includes simultaneously harvesting additional energy based on reactions of a second harvesting component of the MPE harvester to a second type of wireless power transfer signal. In accordance with the described aspects, the second harvesting component is integral with the first harvesting component. By way of example, the second harvesting component 204 is integral with the first harvesting component 202. The first and second harvesting components 202, 204 may be integrated, for instance, using a semiconductor process or by bonding the first and second harvesting components 202, 204. The first and second harvesting components 202, 204 may correspond to and be arranged like the photodiode 404 and the piezo cantilever 402 of FIG. 4, for example.

Regardless of the particular configuration, the second harvesting component 204 of the MPE harvester 106 reacts to a second type of wireless power transfer signal. In particular, the second harvesting component 204 reacts to the second type of wireless power transfer signal simultaneously while the first harvesting component 202 reacts to the first type of wireless power transfer signal. The reactions of the second harvesting component 204 are also effective to generate current at the MPE harvester 106—thereby harvesting energy from the second type of wireless power transfer signal to generate power. The energy harvested by the second harvesting component 204 from the second type of wireless power transfer signal is in addition to the energy harvested by the first harvesting component 202 from the first type of wireless power transfer signal. Further, the second harvesting component 204 harvests the energy at 504 simultaneously while the first harvesting component 202 harvests the energy at 502. In accordance with one or more aspects, the MPE transmitter 108 transmits the second type of wireless power transfer signal, which also passes through bodily tissue of the person 102 to reach the second harvesting component 204. As described in more detail above, the MPE transmitter 108 can transmit this second type of wireless power transfer signal in addition to the first type of wireless power transfer signal. In some implementations, however, the first and second types of wireless power transfer signals may be transmitted by separate wireless power transmitting devices.

At 506, the method includes converting the harvested energy and the additional harvested energy to electrical power that is usable by an electronic device. By way of example, the converter 120 converts the energy harvested by the MPE harvester 106 to electrical power that is usable by the electronic device 104. In some aspects, the converter 120 may change (e.g., increase) a voltage of the power generated to match a voltage used by the electronic device 104. The converter 120 may also convert the power generated in other ways such as converting current from one form to another, e.g., inverting direct current (DC) to alternating current (AC), rectifying AC to DC, and so on. The converter 120 may convert the power generated in still other ways without departing from the spirit or scope of the techniques described herein.

At 508, the method includes transferring the electrical power to the electronic device. By way of example, the power is transferred from the MPE harvester 106 to the electronic device 104 for use via the power coupling 208. As discussed above, the power coupling 208 may be configured as a wired or wireless connection, which may be implemented within the electronic device 104 in scenarios where the MPE harvester 106 is incorporated therein or may be implemented between the electronic device 104 and the MPE harvester 106 when separate devices. In any case, the electrical power may be transferred across a wired connection from the MPE harvester 106 to the electronic device 104. Alternately, the electrical power may be transferred wirelessly from the MPE harvester 106 to the electronic device 104.

At 510, the method includes operating the electronic device using the electrical power. By way of example, the electronic device 104 carries out the functionality for which it is designed using power received over the power coupling 208 from the MPE harvester 106. When the electronic device 104 is an implant for releasing metered doses of medication, for instance, a metered dose of medication is released. Alternately, the electronic device 104 stimulates bodily tissue (e.g., nerves), monitors specific biochemical conditions, and so forth. Although this method step is described with reference to operations performed by biomedical implants, the operations for some implants may correspond to non-medical functionality, such as location tracking, data storage/communication, personal information access, and so on.

FIG. 6 illustrates an example method 600 of configuring a system to harvest energy, for powering an implant, from multiple different wireless power transfer signals. In the following discussion, the MPE harvester 106 or other entities of the example environment 100 may provide means for implementing one or more of the operations described.

At 602, the method includes disposing a first harvesting component in a multiphysics energy (MPE) harvester to harvest energy based on reactions to a first type of wireless power transfer signal. By way of example, consider again FIG. 2, which illustrates the MPE harvester 106 of FIG. 1 in more detail in accordance with one or more aspects generally at 200. In aspects, the first harvesting component 202 is included as part of the MPE harvester 106. In particular, the first harvesting component 202 is disposed in the MPE harvester 106 to harvest energy based on reactions to a first type of wireless power transfer signal.

At 604, the method includes disposing a second harvesting component in the MPE harvester and integral with the first harvesting component to simultaneously harvest additional energy based on reactions to a second type of wireless power transfer signal. By way of example, the second harvesting component 204 is included as part of the MPE harvester 106. In particular, the second harvesting component 204 is disposed in the MPE harvester 106 to harvest energy based on reactions to a second type of wireless power transfer signal, which is different than the first type of wireless power transfer signal. The second harvesting component 204 is included in the MPE harvester 106 to harvest this energy in addition to the energy the first harvesting component 202 is included in the MPE harvester 106 to harvest. Further, the second harvesting component 204 is disposed in the MPE harvester 106 such that the second harvesting component 204 is integral with the first harvesting component 202. To do so, the second harvesting component 204 and the first harvesting component 202 can be integrated in a variety of different ways, such as using a semiconductor process, bonding the components, and so forth.

At 606, the method includes coupling the power-conversion circuitry with the MPE harvester to convert and combine the harvested energy and the additional harvested energy into electrical power. By way of example, the first harvesting component 202 is coupled to the rectifier 306, which may be coupled to the DC/DC converter 308. Further, the second harvesting component 204 may be coupled to the DC/DC converter 310. The rectifier 306 and the DC/DC converters 308, 310 convert and combine the harvested energy and the additional harvested energy to power the implant circuitry 312 or charge a battery (e.g., the power storage 122).

At 608, the method includes coupling the MPE harvester to an electronic device to transfer the electrical power to the electronic device. By way of example, the power coupling 208 is established between the MPE harvester 106 and the electronic device 104. The power coupling 208 allows power to be transferred from the MPE harvester 106 to the electronic device 104, e.g., across wire or wirelessly.

### System-on-Chip

FIG. 7 illustrates an example system-on-chip 700, which includes components capable of implementing aspects of multiphysics energy harvesting for implants. The system-on-chip 700 may be implemented as, or in, any suitable electronic device, such as implants inserted into a body to carry out corresponding functionality or any other device that may utilize power generated using energy harvested by a multiphysics energy harvester.

The system-on-chip 700 may be integrated with, a microprocessor, storage media, I/O logic, data interfaces, logic gates, a transmitter, a receiver, circuitry, firmware, software, or combinations thereof to provide communicative or processing functionalities. The system-on-chip 700 may include a data bus (e.g., cross bar or interconnect fabric) enabling communication between the various components of the system-on-chip. In some aspects, components of the system-on-chip 700 may interact via the data bus to implement aspects of multiphysics energy harvesting for implants.

In this particular example, the system-on-chip 700 includes processor cores 702, system memory 704, and cache memory 706. The system memory 704 or the cache memory 706 may include any suitable type of memory, such as volatile memory (e.g., DRAM), non-volatile memory (e.g., Flash), and the like. The system memory 704 and the cache memory 706 are implemented as a storage medium, and thus do not include transitory propagating signals or carrier waves. The system memory 704 can store data and processor-executable instructions of the system-on-chip 700, such as operating system 708 and other applications. The processor cores 702 execute the operating system 708 and other applications from the system memory 704 to implement functions of the system-on-chip 700, the data of which may be stored to the cache memory 706 for future access. The system-on-chip 700 may also include I/O logic 710, which can be configured to provide a variety of I/O ports or data interfaces for inter-chip or off-chip communication.

The system-on-chip 700 also includes the converter 120, the power storage 122, and implant-specific circuitry 712, which may be embodied separately or combined with other components described herein. For example, the converter 120 and the power storage 122 may be integral with the MPE harvester 106 as described with reference to FIG. 1. The implant-specific circuitry 712 can be implemented to carry out functionality specific to an implant.

The implant-specific circuitry 712 may also be integrated with other components of the system-on-chip 700, such as the cache memory 706, a memory controller of the system-on-chip 700, or any other signal processing, modulating/demodulating, or condition sections within the system-on-chip 700. The implant-specific circuitry 712 and other components of the system-on-chip 700 may be implemented as hardware, fixed-logic circuitry, firmware, or a combination thereof that is implemented in association with the I/O logic 710 or other signal processing circuitry of the system-on-chip 700.

Although subject matter has been described in language specific to structural features or methodological operations, it is to be understood that the subject matter defined in the appended claims is not necessarily limited to the specific features or operations described above, including not necessarily being limited to the organizations in which features are arranged or the orders in which operations are performed.

What is claimed is:

1. An apparatus for harvesting energy simultaneously from multiple different types of wireless power transfer signals to power an implant in a body, the apparatus comprising:
a multiphysics energy (MPE) harvester configured to harvest energy from a first and second type of wireless power transfer signal using:
a first harvesting component configured to react to the first type of wireless power transfer signal effective to harvest the energy from the first type of wireless power transfer signal; and
a second harvesting component that is integral with the first harvesting component and configured to react to the second type of wireless power transfer signal simultaneously as the first harvesting component reacts to the first type of wireless power transfer signal, reactions of the second harvesting component effective to harvest the energy from the second type of wireless power transfer signal.

2. The apparatus as recited in claim 1, further comprising a coupling configured to transfer power from the MPE harvester to the implant in the body, the power generated from the energy harvested by the MPE harvester.

3. The apparatus as recited in claim 2, wherein the MPE harvester and the coupling are incorporated within the implant.

4. The apparatus as recited in claim 2, wherein the MPE harvester is in the body, remote from the implant and the coupling connects the MPE harvester and the implant for the power transfer.

5. The apparatus as recited in claim 4, wherein the coupling is a wired coupling between the MPE harvester and the implant.

6. The apparatus as recited in claim 4, wherein the coupling is a wireless coupling between the MPE harvester and the implant.

7. The apparatus as recited in claim 1, wherein the first harvesting component is formed of a piezoelectric material and the first type of wireless power transfer signal is an acoustic signal.

8. The apparatus as recited in claim 1, wherein the second harvesting component is a photodiode and the second type of wireless power transfer signal is an infrared signal.

9. The apparatus as recited in claim 1, wherein the first harvesting component is a piezo cantilever array and the second harvesting component is a photodiode that is stacked on the piezo cantilever array to serve as a proof mass for the piezo cantilever array.

10. The apparatus as recited in claim 1, wherein at least one of the first harvesting component or the second harvesting component is configured to have a microelectromechanical system (MEMS) form factor.

11. The apparatus as recited in claim 1, wherein the second harvesting component is integrated with the first harvesting component via at least one of a semiconductor process or by bonding the second harvesting component to the first harvesting component.

12. The apparatus as recited in claim 1, further comprising power-conversion circuitry to convert and combine the harvested energy harvested using the first and second harvesting components into electrical power that is usable by the implant.

13. A method for harvesting energy from multiple different types of wireless power transfer signals simultaneously to power an electronic device in a body, the method comprising:
harvesting energy based on reactions of a first harvesting component of a multiphysics energy (MPE) harvester within the body to a first type of wireless power transfer signal;
simultaneously harvesting additional energy based on reactions of a second harvesting component of the MPE harvester to a second type of wireless power transfer signal that is different from the first type of wireless power transfer signal, the second harvesting component being integral with the first harvesting component; and
converting the harvested energy and the additional harvested energy into electrical power that is usable by the electronic device.

14. The method as recited in claim 13, further comprising rectifying the harvested energy harvested using one of first or second harvesting components.

15. The method as recited in claim 13, further comprising transferring the electrical power to the electronic device.

16. The method as recited in claim 15, wherein the electrical power is transferred to the electronic device over a wired coupling between the MPE harvester and the electronic device.

17. The method as recited in claim 15, wherein the electrical power is transferred to the electronic device over a wireless coupling between the MPE harvester and the electronic device.

18. The method as recited in claim 13, further comprising operating the electronic device using the electrical power from the harvested energy and the additional harvested energy.

19. The method as recited in claim 13, further comprising simultaneously harvesting further additional energy based on reactions of at least a third harvesting component of the MPE harvester, the third harvesting component being integral with at least one of the first or second harvesting components.

20. The method as recited in claim 19, wherein the reactions of the at least third harvesting component are to one of the first or second wireless power transfer signals.

21. The method as recited in claim 19, wherein the reactions of the at least third harvesting component are to at least a third type of wireless power transfer signal that is different from the first and second type of wireless power transfer signal.

22. A method for configuring a system to harvest energy from multiple different types of wireless power transfer signals simultaneously to power an implant, the method comprising:
disposing a first harvesting component in a multiphysics energy (MPE) harvester to harvest energy based on reactions to a first type of wireless power transfer signal;
disposing a second harvesting component in the MPE harvester and integral with the first harvesting component to simultaneously harvest additional energy based on reactions to a second type of wireless power transfer signal different from the first type of wireless power transfer signal; and
coupling power-conversion circuitry with the MPE harvester to convert and combine the harvested energy and the additional harvested energy into electrical power that is usable by the implant.

23. The method as recited in claim 22, further comprising coupling the MPE harvester to the implant to transfer the electrical power to the implant.

24. The method as recited in claim 22, wherein the power-conversion circuitry is incorporated as part of the MPE harvester.

25. The method as recited in claim 22, wherein disposing the second harvesting component in the MPE harvester includes attaching the second harvesting component to the first harvesting component using a semiconductor process or by bonding the second harvesting component to the first harvesting component.

26. The method as recited in claim 22, further comprising incorporating the MPE harvester and the power-conversion circuitry in the implant.

27. The method as recited in claim 22, further comprising configuring at least one of the first harvesting component or the second harvesting component to have a microelectromechanical system (MEMS) form factor.

28. An apparatus for powering an implant using energy harvested simultaneously from multiple different types of wireless power transfer signals, the apparatus comprising:
a first harvesting means for harvesting energy from a first type of wireless power transfer signal;
a second harvesting means that is integral with the first harvesting means for simultaneously harvesting additional energy from a second type of wireless power transfer signal; and
a power generation means for generating power that is usable by the implant from the energy and the additional energy harvested by the first and second harvesting means, respectively.

29. The apparatus as recited in claim 28, further comprising a power transfer means for transferring the power generated by the power generation means to the implant.

30. The apparatus as recited in claim 28, wherein:
the first harvesting means harvests the energy from one or more acoustic signals; and
the second harvesting means harvests the additional energy from electromagnetic radiation.
